(19) United States
(12) Patent Application Publication
(10) Pub. No.: US 2004/0209850 A1
(43) Pub. Date: Oct. 21, 2004

(54) Methods of treating pain and compositions for use therefor

(75) Inventor: Najib Babul, Norristown, PA (US)

Correspondence Address: STERNE, KESSLER, GOLDSTEIN & FOX PLLC, 1100 NEW YORK AVENUE, N.W., WASHINGTON, DC, 20005, US

(73) Assignee: TheraQuest Biosciences, LLC, Blue Bell, PA

(21) Appl. No.: 10/824,357

(22) Filed: Apr. 15, 2004

Related U.S. Application Data
(60) Provisional application No. 60/462,691, filed on Apr. 15, 2003.

(51) Int. Cl. A61K 31/60; A61K 31/415; A61K 31/365

(52) U.S. Cl. 514/165; 514/282; 514/406; 514/471; 514/570; 514/569

(57) ABSTRACT

The present invention relates to a method of treating or preventing pain and to pharmaceutical compositions useful for carrying out said methods. The present invention is directed to a method of treating or preventing pain comprising administering a selected analgesic and a beta adrenergic agonist to a subject in need of such treatment, wherein said beta adrenergic agonist produces an enhanced effect of said analgesic. The present invention is also directed to pharmaceutical compositions comprising an analgesic and a beta adrenergic agonist useful for carrying out the method of the present invention.

This application claims the benefit of U.S. Provisional Application No. 60/462,691, filed Apr. 15, 2003, which is fully incorporated herein by reference.

### BACKGROUND OF THE INVENTION

1. Field of the Invention

The present invention is in the field of pharmaceutical compositions and the use thereof for treating and preventing pain.

2. Background Art

The medical condition of pain is a complex physiological process that involves a number of sensory and neural mechanisms. Pain can be defined as an unpleasant sensory and emotional experience associated with actual or potential tissue damage, or described in terms of such damage.

Pain is most often classified by time course or mechanism as acute pain, inflammatory pain, visceral pain, breakthrough pain, neuropathic pain, chronic pain, or cancer-related pain. Acute pain is a normal, predictable physiological response to an adverse chemical, thermal, or mechanical stimulus associated with surgery, trauma, or acute illness. It is normally self-limited. When the condition producing the pain resolves, the pain goes away. Acute pain includes post-surgical pain, post-traumatic pain, renal colic, and headache. Acute pain may be experienced as a single event, or it may be episodic. Chronic pain is usually defined as pain persisting longer than the expected time of tissue healing. Injury or a disease process can trigger chronic pain, but other factors besides the triggering event can perpetuate the pain. For example, the nervous system itself may be damaged by the initial injury and be unable to return to normal function despite healing of the injury itself. In some cases, the precise cause of chronic pain may be unknown (idiopathic pain), but the pain may still respond to the treatment. Chronic pain includes such syndromes as low back pain, myofascial pain, osteoarthitis, neuropathic pain, fibromyalgia and inflammatory pain states such as rheumatoid arthritis. Cancer-related pain is due to the primary tumor itself, metastatic disease, paraneoplastic syndromes, or effects of cancer treatment. Cancer-related pain can include elements of both chronic pain and acute pain. Neuropathic pain is secondary to injury to nerves and includes postherpetic neuralgia, diabetic neuropathy, postamputation pain, mono- and polyneuropathies, radiculopathy, and central pain.

Management of pain, and particularly chronic pain, is complex and frequently unsuccessful or only partially successful. However, rarely do physicians engage in aggressive pain management. Undertreatment for pain frequently leads to conditions where patients are forced to suffer pain up to the point of tolerability before receiving medication, and the medication is usually only partially effective. Ineffective pain management is a consequence of lack of training, and of fear of narcotics on the part of patients, the medical personnel, and society in general. Children, because of their natural reticence and budding communication skills combined with a greater fear of over-administering “dangerous” narcotics particularly suffer from under treatment for pain.

Moreover, rapid tolerance and marked resistance to narcotics frequently develop, thus rendering many analgesic agents ineffective (see, e.g., Abram, Reg. Anesth. 18(SUPPL):406-413 (1993)).

Problematic is the possibility of adverse side effects, particularly gastric distress that accompanies oral administration, or the fear that injections can inspire.

Frequently, a patient suffering from chronic pain will require medication to control stomach and other gastric problems as a result of oral administration of drugs. Alternatives to oral self-administration for most of the analgesic and sedative medications for the treatment of chronic pain are not common, can be cumbersome (e.g., i.v. or s.c. administration requires use of a cannula or needle), and generally require medical training.

Currently, medical practitioners may choose from several well-accepted classes of pharmaceutical agents in their attempts to alleviate and prevent pain. The most commonly used agents include nonsteroidal antiinflammatory agents (NSAIDs), e.g., aspirin, ibuprofen, ketoprofen, diclofenac; opioids, e.g., morphine, hydromorphene, hydrocodone, oxycodone, tramadol, and codeine; cyclooxygenase-2 (COX-2) inhibitors, e.g., celecoxib, valdecoxib, and rofecoxib; acetaminophen; tramadol; tricyclic antidepressants, e.g., amitriptyline and despiramine; and antiepileptics, e.g., gabapentin, oxcarbazepine, and lamotrigine.

Of the many challenges that occur when pharmacologically treating any disease or pathological condition, including pain, alleviating the symptoms without causing counterproductive side effects is often the greatest. This challenge presents itself when medical practitioners use medicinal agents to treat pain. Although the aforementioned pharmacological classes are frequently effective for the treatment of certain types of pain, the chronic and acute use of these analgesic agents produces a number of significant, undesirable side effects.

The opioids are well-known for their potential for physical dependence and addiction. Dependence appears to be related to the dose of opioid taken and the period of time over which it is taken by the subject. Additionally, chronic use of morphine or other opioids can lead to tolerance, a result that reduces the effectiveness of the analgesic opioid. Other side effects of opioids include nausea, vomiting, constipation, sedation, and potentially fatal respiratory depression. When a subject is tolerant to opioid narcotics, increased doses are required to achieve a satisfactory analgesic effect. For this reason, alternative therapies for the management of chronic pain are widely sought. In addition, compounds which serve as either a replacement for or as an adjunct to opioid treatment in order to decrease the dosage of analgesic compound have utility in the treatment of pain.

The non-steroidal anti-inflammatory drugs (NSAIDs), as a class, are usually only effective against pain of low to moderate intensity. Moreover, long term use of many NSAIDs produce gastrointestinal side-effects such as ulceration and bleeding. The main mechanism by which NSAIDs exert an analgesic effect is through the inhibition of the synthesis of certain prostaglandin, or prostanoids. The synthesis of prostanoids utilizes two distinct cyclooxygenase (COX) enzymes: COX-1 and COX-2. Traditional NSAIDs inhibit both enzymes. NSAIDs may also inhibit other lipogenic enzymes, such as 5-lipooxygenase. Although NSAIDs are not addictive, they are not without significant toxic effects, such as gastrointestinal injury hepatotoxicity and decrease clotting ability. Additionally, the NSAIDs are also not very useful in treating severe pain.

Acetaminophen can cause liver and kidney toxicity, especially at higher doses and when given for long periods.

Given that significant drawbacks and side effects accompany the use of currently prescribed analgesic agents, there is a need for new therapeutic methods and pharmaceutical compositions that have analgesic activity. More specifically, there is a need for new analgesic methods and pharmaceutical compositions that have reduced side effects, enhanced analgesic effects, better therapeutic indices, and/or faster onset of action.

One approach to the development of new analgesic methods and compositions is combining a known analgesic drug with a second pharmaceutical agent, wherein the second pharmaceutical agent enhances the activity of the known analgesic drug. Examples include U.S. Pat. No. 4,558,051, in which a particular xanthine derivative is used to enhance the analgesic effect of an NSAID or a narcotic analgesic; U.S. Pat. No. 5,834,479, in which an NMDA receptor blocker is used to enhance the analgesic effect of an NSAID; and U.S. Pat. No. 6,204,271, in which moxonidine is used to enhance the analgesic activity of an opioid analgesic drug.

U.S. Pat. Nos. 4,965,065; 5,043,358; and 5,071,842, describe the use of NSAIDs with beta adrenergic agonists is disclosed. NSAIDs disclosed as being useful include salicylates, e.g., aspirin; propionic acid derivatives, e.g., ibuprofen and naproxen; fenamates, e.g., mefanamic acid, meclofenamate sodium, diclofenac, and diclofenac sodium; indole derivatives, e.g., indomethacin; pyrroleakanoic acid derivatives, e.g., tolmetin; pyrazalone derivatives, e.g., phenylbutazone; and oxicams, e.g., piroxicam. The combined use of an NSAID with a beta adrenergic agonist is said to be beneficial for protecting against gastrointestinal injury induced by NSAIDs, inhibiting gastric acid secretion, and treating peptic ulcers. However, the art does not suggest that beta adrenergic agonists are useful for enhancing the analgesic potency of NSAIDs or that beta adrenergic agonists reduce other side effects of NSAIDs such as hepatotoxicity. Moreover, the art does not suggest that beta adrenergic agonists are useful for enhancing the analgesic effect of pain relieving drugs other than NSAIDs.

Furthermore, a need exists for developing a method of enhancing the analgesic effect of opioids, e.g., morphine. By enhancing the effect of an opioid, one could administer a lower dose of the opioid to achieve the desired analgesic effect and, at the same time, reduce side effects and/or decrease the potential for physical addition.

### BRIEF SUMMARY OF THE INVENTION

Surprisingly, selected analgesics can be advantageously used together with beta adrenergic agonists and administered to animals to not only elicit a more potent analgesic response but also to evoke such response more rapidly and/or for longer duration than possible by administration of the analgesic agent alone.

A first aspect of the present invention directed to a novel method for eliciting an analgesic response, said method comprising administering an effective amount of an analgesic drug and an analgesic-enhancing amount of a beta adrenergic agonist.

A second aspect of the present invention is directed to a novel method for eliciting an analgesic response, said method comprising administering an effective analgesic amount of an analgesic and an amount of a beta adrenergic agonist sufficient to hasten the onset of the analgesic response or to enhance the analgesic response.

A third aspect of the present invention is directed to a novel method for eliciting an analgesic response, said method comprising administering an subanalgesic amount of an analgesic and an amount of a beta adrenergic agonist.

A fourth aspect of the present invention is directed to a novel method for increasing the duration of analgesic response, said method comprising administering an effective analgesic amount of an analgesic and an amount of a beta adrenergic agonist sufficient to increase the duration of effect of the analgesic.

A fifth aspect of the present invention is directed to novel pharmaceutical compositions of matter for use in eliciting an analgesic or anti-inflammatory response comprising an effective analgesic amount of a selected analgesic and an amount of a beta adrenergic agonist sufficient to hasten the onset of or enhance the analgesic response.

A sixth aspect of the present invention is directed to a novel method for reducing the tolerance to the analgesic effects of opioids, said method comprising administering an effective analgesic amount of an analgesic and an amount of a beta adrenergic agonist sufficient to reduce analgesic tolerance.

A seventh aspect of the present invention is directed to a novel method for reducing the dependence to opioid analgesics, said method comprising administering an effective analgesic amount of an analgesic and an amount of a beta adrenergic agonist sufficient to reduce opioid dependence.

### DRAWINGS

FIG. 1. The dose-effect curves for albuterol (TQ-1016) and for morphine are shown as a function of percent maximal possible effect (% MPE). Albuterol did not demonstrate dose-dependent antinociception. The highest effective dose was 1 mg/kg, with decreasing effect becoming evident as dose increased. The effect of albuterol approximated the effect of the lowest (0.3 mg/kg to 2.5) doses of morphine. Morphine demonstrated dose-dependent antinociception. Dose-dependency was evident between 2.5 mg/kg and 10 mg/kg, and represents the linear part of the dose-response curve.

FIG. 2. The antinociceptive dose-response curves for morphine and for morphine in the presence of albuterol (TQ-1016) are shown for data collected 30 minutes and 60 minutes after injection. The dose-effect curves for morphine in the presence of albuterol are significantly shifted to the right of the morphine dose-response curve alone in both sets of data. These results demonstrate a synergistic antinociceptive interaction.

FIG. 3 show the effect of albuterol on chemically induced abdominal constrictions in the mouse.

FIG. 4 shows the effect of indomethacin on chemically induced abdominal constrictions in the mouse.

FIG. 5 shows the effect of co-administration of albuterol and indomethacin on chemically-induced abdominal constrictions in the mouse.

### DETAILED DESCRIPTION OF THE INVENTION

The present invention provides a method of treating or preventing pain comprising administering to a subject in need of pain treatment or pain prevention (a) one or more analgesics and (b) one or more beta adrenergic agonists. In one embodiment, the method comprises administering one analgesic and one beta adrenergic agonist. The method produces an enhanced analgesic effect of said analgesic.

In one embodiment, said one or more beta adrenergic agonists are administered in an amount which enhances the effect of the analgesic. In one embodiment of the present invention, “enhanced effect” means that, when coadministered with a beta adrenergic agonist, lower doses of the selected analgesic are required to achieve the same analgesic effect as when the analgesic is administered alone or greater analgesic effect is achieved when usual doses of the selected analgesic are administered with a beta adrenergic. For example, across all doses, a greater analgesic response is achieved with coadministration of an analgesic with a beta adrenergic agonist when compared with administration of the analgesic by itself.

The invention also provides a method of treating or preventing pain by administering one or more analgesics with one or more beta adrenergic agonists wherein the amount of said beta adrenergic agonist hastens the onset of action of the analgesic.

The invention also provides a method of treating or preventing pain by administering one or more analgesics with one or more beta adrenergic agonists wherein the amount of said beta adrenergic agonist increases the duration of the activity of said analgesic.

The invention also provides a method for reducing the tolerance to the analgesic effects of opioid analgesics by administering one or more opioid analgesics with one or more beta adrenergic agonists wherein the amount of said beta adrenergic agonist decreases the analgesic tolerance to said opioid.

The invention also provides a method for reducing the narcotic dependence to opioid analgesics by administering one or more opioid analgesics with one or more beta adrenergic agonist wherein the amount of said beta adrenergic agonist decreases the narcotic dependence of said opioid.

The invention further provides a method of alleviating or preventing pain in a mammal in need of pain treatment or pain prevention by administering one or more analgesics with one or more beta adrenergic agonists. In one embodiment, the mammal in need of pain treatment or pain prevention is a human.

The invention further provides a method of alleviating or preventing pain in a mammal in need of pain treatment or pain prevention by administering at least one analgesic and at least one beta adrenergic agonist as a single pharmaceutical composition. Said analgesic and said beta adrenergic agonist can also be administered as separate pharmaceutical compositions. In one embodiment, said analgesic and said beta adrenergic agonist are coadministered as a sustained release dosage form.

Also provided is a composition comprising (a) a pain-alleviating or pain-preventing amount of one or more analgesics, wherein said analgesic is selected from the group consisting of acetaminophen, NSAID, COX-2 inhibitor, opioid, and tramadol, and (b) one or more beta adrenergic agonists. In one embodiment, said one or more beta adrenergic agonists are administered in an amount which enhances the activity of said analgesic. In one embodiment, said one or more beta adrenergic agonists are administered in an amount which hastens the onset of activity of said analgesic. In one embodiment, said one or more beta adrenergic agonists are administered in an amount which increases the duration of the activity of said analgesic.

In one embodiment, the compositions of the present invention comprise one or more excipients and one or more inert carriers.

When a selected analgesic is combined with a beta adrenergic agonist in accord with the present invention, the beta adrenergic agonist produces an enhanced activity of said analgesic. The enhanced activity of said analgesic may be one or more of the following:

(1) the analgesic effect of the selected analgesic has a faster onset of action;

(2) the analgesic effect of the selected analgesic has a greater overall effect;

(3) a lower dose of the selected analgesic is required for the same analgesic effect as when the analgesic is administered alone;

(4) across all doses, a greater analgesic response is achieved;

(5) the analgesic effect of the selected analgesic has a longer duration of action;

(6) an increase peak analgesic effect;

(7) a sub-therapeutic analgesic dose produces a therapeutic analgesic effect; and

(8) the reduction and/or elimination of one or more side effects caused by the analgesic, provided that the NSAID-induced gastrointestinal injury is not included.

When the analgesic is an opioid, the enhanced analgesic activity may additionally be or include one or more of the following:

(1) there is decreased tolerance or development of tolerance to the analgesic effects of an opioid analgesic;

(2) there is a reduced dependence or development of dependence to opioid analgesics.

With respect to the enhanced activity of the analgesic, it is meant that the method of the present invention will produce an enhanced activity of the analgesic when compared to administering an analgesic alone.

In one embodiment of the present invention, administration of a beta adrenergic agonist with an certain analgesic produces a reduction in one or more side effects of the analgesic. Such side effects may include nausea, vomiting, constipation, drowsiness, sedation, dizziness, fatigue, dry mouth, addiction, physical dependence, psychological dependence, tolerance, somnolence, headache, sweating, emesis, and pruritus. It is also understood that NSAID-induced gastrointestinal injury is excluded. Thus, benefits described in U.S. Pat. Nos. 4,965,065; 5,043,358; and 5,071,842 are excluded from the present invention.

For patients suffering pain, and most especially for patients suffering severe pain, the time from administration of medication to the onset of effective relief is clearly of paramount importance. Accordingly, in one embodiment of the present invention, a beta adrenergic agonist is used to shorten the onset time (i.e., substantially hasten the onset) of analgesia when embodied with an analgesic.

Further, the ability of such beta adrenergic agonists to enhance the effects of certain analgesics, e.g., to substantially reduce the amount of selected analgesic which is required to elicit a given analgesic response, is also an unexpected and important embodiment of the present invention, and permits the use of the selected analgesic in quantities substantially less than the dosages presently suggested as an analgesic agent in humans. Use of lower doses lowers the incidence and/or severity of undesirable side effects, such as lessening addiction potential. Moreover, at a given dosage level, a greater analgesic response can be achieved compared to administration of the analgesic alone.

The selected analgesic/beta adrenergic agonist compositions of the present invention are also advantageous in that the use of a beta adrenergic agonist counteracts the effects of the selected analgesic such that the patient is more alert, has better motor skills, and/or, in certain instances, hosts an improved sense of well-being as compared to when the analgesic is administered alone.

For example, an opioid analgesic, such as oxycodone, codeine, morphine, and hydromorphone, can be administered with a beta adrenergic agonist according to the methods of the present invention. In adult humans, the dose of narcotic required varies considerably, depending on pain severity, tolerability of side effects, tolerance to analgesic effects, and patient response to treatment. The typical effective analgesic amounts, for use in unit doses, of analgesic/beta adrenergic agonist compositions of the present invention, to be administered every 4 to 6 hours as needed by the oral route, are about 5 to 120 mg morphine, 0.5 to 24 mg hydromorphone hydrochloride, about 15 to 120 mg codeine sulfate or phosphate, about 2.5 to 60 mg oxycodone hydrochloride, about 1 to 5 mg levorphanol tartrate, about 50 to 100 mg meperidine hydrochloride, about 65 to 200 mg propoxyphene hydrochloride or napsylate, about 2 to 30 mg methadone hydrochloride, about 25 to 150 mg propiram fumarate, about 0.15 to 8 mg buprenorphine hydrochloride, about 25 to 100 mg pentazocine hydrochloride, about 0.5 to 10 mg butophanol tartrate, about 25 to 400 mg tramadol, and about 100 to 400 mg meptazinol hydrochloride.

The amount of beta adrenergic agonist in the analgesic composition will be an amount sufficient to shorten the onset time and/or to enhance analgesia. For humans, an average unit dosage analgesic composition will typically contain from about 0.001 mg to about 400 mg beta adrenergic agonist. The daily analgesic does in humans will vary with the selected narcotic analgesic, and may of course be as low as the amount contained in a single unit does as set forth above. The daily dose for use in the treatment of moderate to severe pain will preferably not exceed 96 mg hydromorphone hydrochloride, or 360 mg codeine sulfate or phosphate, or 240 mg oxycodone hydrochloride or hydrochloride/terephthalate mixture, or 40 mg levorphanol tartrate, or 600 mg meperidine hydrochloride, or 400 mg propoxyphene hydrochloride or napsylate, or 120 mg methadone hydrochloride, or 600 mg propiram fumarate, or 60 mg buprenorphine hydrochloride, or 400 mg pentazocine hydrochloride, or 80 mg nalbuphine hydrochloride, or 40 mg butophanol tartrate, or 600 mg tramadol hydrochloride, and 2000 mg of the beta adrenergic agonist, although greater amounts could be employed if tolerated by the patient.

The typical effective analgesic amounts of the opioid dose varies depending on the route of administration and the desired effect. Moreover, there are established standards for analgesic equivalence among different opioids. In one embodiment, the method of the invention comprises administering analgesic/beta adrenergic agonist compositions, to be administered every 4 to 6 house as needed at doses of equivalent to oral morphine 5 mg to 120 mg, oral oxycodone 2.5 to 60 mg, oral hydromorphone 0.5 to 16 mg, parenteral morphine 0.5 to 20 mg, parenteral hydromorphone 0.1-4 mg, and, in the case of other opioid analgesics, including those noted in Table 1 below, doses providing analgesic effects equivalents to the doses provided herein, based on acceptable standard references.

Definitions

“Drug,” “pharmacological agent,” “pharmaceutical agent,” “active agent,” and “agent” are used interchangeably and are intended to have their broadest interpretation as to any therapeutically active substance which is delivered to a living organism to produce a desired, usually beneficial effect. In general, this includes therapeutic agents in all of the major therapeutic areas, also including proteins, peptides, oligonucleotides, and carbohydrates as well as inorganic ions, such as calcium ion, lanthanum ion, potassium ion, magnesium ion, phosphate ion, and chloride ion.

“Pharmaceutically or therapeutically acceptable excipient or carrier” refers to a substance which does not interfere with the effectiveness or the biological activity of the active ingredients and which is not toxic to the hosts, which may be either humans or animals, to which it is administered. Pharmaceutically or therapeutically acceptable excipients or carriers are well known in the art.

“Therapeutically effective amount” refers to the amount of an active agent sufficient to induce a desired biological result. That result may be alleviation of the signs, symptoms, or causes of a disease, or any other desired alteration of a biological system.

The phrase “therapeutically-effective” is intended to qualify the amount of each agent which will achieve the goal of improvement in disease severity and the frequency of incidence over treatment of each agent by itself, while avoiding adverse side effects typically associated with alternative therapies.

The term “effective amount” means the quantity of a compound according to the invention necessary to prevent, to cure, or at least partially arrest a symptom of local pain or discomfort in a subject. A subject is any animal, preferably any mammal, more preferably a human. Amounts effective for creating a substantially local therapeutic effect will, of course, depend on the severity of the disease causing the painful condition, and the weight and general state of the subject. Typically, animal models, such as those described in the Background and Examples herein, may be used to determine suitable dosages to be used. A recent pain scale developed by Galer et al., Neurology 48:332-338 (1997)), which uses terminology specific for neuropathic pain, should be better able to delineate the symptoms within the syndrome. In addition, various general considerations taken into account in determining the “therapeutically effective amount” are known to those of skill in the art and are described, e.g., in Gilman et al., eds., Goodman And Gilman's The Pharmacological Bases of Therapeutics, 8th ed., Pergamon Press (1990); and Remington's Pharmaceutical Science, 17th ed., Mack Publishing Co., Easton, Pa. (1990), each of which is herein incorporated by reference.

The term “beta adrenergic agonist,” as used herein, refers to a drug that activates a beta adrenergic receptor. The terms “beta adrenergic agonist” and “beta agonist,” as used herein, are synonymous. A beta adrenergic agonist may be a selective beta-1 adrenergic agonist, a selective beta-2 adrenergic agonist, or a mixed beta-1/beta-2 adrenergic agonist. The term “mixed beta-1/beta-2 agonist,” as used herein, refers to a drug that activates both the beta-1 receptor and a beta-2 receptor.

The term “activate” or grammatical variants thereof, as used herein, refers to binding to a receptor and causing the receptor to produce a cellular or physiological change. For example, in one embodiment, a drug that activates a beta adrenergic receptor will cause an increase in the intracellular level of cyclic adenosine monophosphate (cAMP).

The term “subject” for purposes of treatment includes any animal subject who has any one of the known forms of pain. The subject is preferably a mammal and more preferably is a human.

As used herein, tolerance refers to: 1) the need to increase the dose of opioid over time in order to achieve the amount of analgesia or euphoria, or 2) the observation that chronic administration of the same dose results in reduced analgesia, euphoria, or other opioid effects. It has been found that a remarkable degree of tolerance develops to the analgesic, respiratory depressant, sedative, emetic, and euphoriant effects of opioids. However, the rate at which this attenuation of effect (tolerance) may develop in either a patient with pain or an addict depends on the pattern of use. If the opioid is used frequently, it may be necessary to increase the dose. Tolerance does not develop equally or at the same rate to all the effects of opioids, and even opioid users who are highly tolerant to respiratory depressant effects of opioids continue to exhibit constipation. Tolerance to opioids largely disappears when the withdrawal syndrome has been completed. The potential for the development of tolerance with chronic opioid therapy is a characteristic feature of all the opioid analgesics and the possibility of developing tolerance is one of the major concerns in the use of opioids for the treatment of pain.

As used herein, dependence refers to “physical dependence” or “psychological dependence.” Physical dependence, characterized by withdrawal symptoms, may develop upon repeated administrations or prolonged use of opioid analgesics. Physical dependence may manifest itself gradually upon cessation of treatment or even following rapid tapering of an opioid analgesic, or it may manifest itself abruptly after (e.g., within 30 minutes) after administration of an opioid antagonist. Depending upon the drug to which dependence has been established and the duration of use and dose, withdrawal symptoms will vary in nature, frequency, duration and intensity. The most common symptoms of the withdrawal syndrome include anorexia, weight loss, nausea, vomiting, pupillary dilation, goose flesh, chills alternating with excessive sweating, abdominal cramps, muscle spasms, hyperirritability, lacrimation, rinorrhea, and increased heart rate. Abstinence syndrome typically begins to occur 24-48 hours after the last dose, and the syndrome reaches its maximum intensity about the third day and may not begin to decrease until the third week. Psychological dependence (i.e., addiction) to opioids is characterized by drug-seeking behavior characterized by use of opioid despite harm, non-medical use of opioids, use outside approved medical supervision, and use for pleasurable effects, e.g., euphoria. The potential for the development of physical dependence with chronic opioid therapy is a characteristic feature of all the opioid analgesics and the possibility of developing psychological dependence (i.e., addiction) is one of the major concerns in the use of opioids for the treatment of pain, even though iatrogenic addiction is infrequent.

As used herein, “equianalgesic doses,” also referred to as “analgesic equivalence,” is a term used by practitioners of the art to refer to approximately comparable doses of analgesics required to provide a similar magnitude of analgesia. There are established standards to allow practitioners of the art to convert the dose of one opioid analgesic, given by any route of administration, to an approximately equivalent dose of another opioid analgesic, given by any route of administration. These analgesic conversion tables provide what in the art is called “analgesic equivalence” or “equianalgesic doses” (Anon. Principles of Analgesic Use in the Treatment of Acute Pain and Cancer Pain, Fourth Edition, 199, American, Pain Society, 1999; Gutstein H B & Akil H. Opioid Analgesics. In: Goodman and Gilman's The Pharmacologic Basis of Therapeutics, 10th Ed., Hardman J G & Limbird LE (Eds), p 569-619, McGraw-Hill, New York, N.Y.). The availability of analgesic equivalence tables allows practitioners of the art to convert patients from one opioid analgesic to another without a protracted titration period on the new analgesic.

Beta-Agonists

The methods and compositions of the present invention utilize a beta adrenergic agonist. Beta adrenergic agonists include mixed beta1/beta2 agonists, selective beta-1 agonists, and selective beta-2 agonists.

Determination if a compound is a beta adrenergic agonist is within the ability of one of ordinary skill in the art. For example, one may utilize the assay as described in U.S. Pat. No. 4,894,219.

Suitable beta adrenergic agonists include, but are not limited to, isoetharine, which is 1-(3,4-dihydroxyphenol)-2-isopropyl amino ethanol hydrochloride; orciprenaline (1-(3,5-dihydroxyphenyl)-2-isopropyl amino ethanol sulphate); reproterol (7-(−3-(propyl-3-5, beta-trihydroxyphenyl)amino)propyl)theophylline); salbutamol (2-tertbutylamino-1-(4-hydroxy-3-hydroxymethylphenyl) ethanol sulphate); terbutaline (2-tert-butylamino-1-(3,5-dihydroxyphenyl) ethanol sulphate); fenoterol(5-[1-hydroxy-2-[[2-(4-hydroxyphenyl)-1-methylethyl]amino]ethyl)-1,3-benzenediol; cimaterol(benzonitrile, 2-amino-5-[1-hydroxy-2-[(1-methylethyl)amino]ethyl]); and ractopamine ([1-(4-hydroxyphenyl)-2-(1-methyl-3-(4-hydroxyphenyl))propylamino]ethanol).

Other suitable beta adrenergic agonists are those compounds disclosed in U.S. Pat. No. 4,600,710.

Other suitable beta adrenergic agonists are bitolterol, broxaterol, clenbuterol, colterol, fenoterol, fomoterol, formoterol, isoetharine, isoproterenol (isoprenaline), isoxsuprine, mabuterol, metaproterenol, orciprenaline, picumeterol, procaterol, reproterol, rimiterol, ritodrine, salbutamol (albuterol), salmeterol, terbutaline, and zinterol.

In another embodiment, the beta adrenergic agonist is used as a racemic mixture. In another embodiment, the beta adrenergic agonist is used as a single stereoisomer. In another embodiment, the beta adrenergic agonist is used as a mixture of stereo isomers containing unequal amounts of stereoisomers.

In one embodiment, the beta adrenergic agonist used in the method is selected from the group consisting of albuterol, salmeterol, terbutaline, and fenoterol. In another embodiment, the beta adrenergic agonist used in the method is selected from the group consisting of albuterol, salmeterol, terbutaline, and isoproteronol. In yet another embodiment, the beta adrenergic agonist used in the method is selected from the group consisting of albuterol, salmeterol, and terbutaline. In another embodiment, the beta adrenergic agonist used in the method albuterol.

Generally, the beta adrenergic agonist is administered in an amount of about 0.001 mg to about 400 mg, every four to six hours. In another embodiment, the beta adrenergic agonist is administered in an amount from about 0.01 to about 40 mg, every four to six hours. In another embodiment, the beta adrenergic agonist is administered in an amount from about 0.1 to about 4 mg, every four to six hours.

The beta adrenergic agonist may be administered on a weight basis as well. In one embodiment, the beta adrenergic agonist is administered in an amount of about 0.0001 mg/kg/day to about 40 mg/kg/day. In another embodiment, the beta adrenergic agonist is administered in an amount of about 0.001 mg/kg/day to about 4 mg/kg/day. In one embodiment, the beta adrenergic agonist is administered in an amount of about 0.01 mg/kg/day to about 0.4 mg/kg/day.

Classes of Analgesics

Several classes of analgesics can be used in the methods and compositions of the present invention. The analgesics for use in the methods and compositions of the present invention can be selected from the following categories:

(1) COX-2 inhibitors;

(2) NSAIDS;

(3) acetaminophen;

(4) tramadol; and

(5) opioids.

NSAIDs

Nonsteroidal anti-inflammatory drugs (NSAIDs) typically have analgesic, anti-inflammatory and antipyretic properties. Their mode of action appears to involve inhibition of cyclooxygenases (COX-1 and COX-2), leukotriene biosynthesis, and antibradykinin activity. Although efficacy with NSAIDs is dose related, there is a “ceiling” to the analgesic effect, i.e., further dose increases do not usually provide a proportional increase in analgesic effect. NSAIDs can produce adverse effects that are usually related to the dose and duration of treatment. Although the exact mechanisms of adverse effects have not been clearly established, at least some appear related to COX-1 inhibition. In addition to its gastrointestinal adverse effects, NSAIDS produce dose related inhibition of platelet aggregation, prolongation of bleeding time, renal impairment and hepatotoxicity.

In another embodiment, the present invention is directed to a method of treating or preventing pain, comprising administering, to a subject in need of such treatment or prevention, a NSAID and albuterol, wherein said albuterol produces an enhanced effect of said analgesic, provided that the enhanced effect does not include reduced NSAID-induced gastrointestinal injury.

COX-2 Selective Inhibitors

It has been proposed that the anti-inflammatory, antipyretic, and analgesic effects of NSAIDs are related to their inhibition of cyclooxygenase-2 (COX-2) enzyme. The unwanted side effects, particularly gastric toxicity, is mainly due to inhibition of COX-1 enzyme. Thus, researchers have developed newer analgesic agents that are selective for COX-2 versus COX-2. These agents have been shown to have safer profile in terms of gastric damage.

However, selective COX-2 analgesic agents are not completely void of side effects. Recent research has shown that COX-2 selective agents reduce the rate of gastric healing and vascular growth. Such side effects can have significant consequences and lead one to look for newer compositions comprising COX-2 selective analgesics that minimize the side effects.

According to the present invention, a COX-2 analgesic is administered with a beta adrenergic agonist to treat or prevent pain.

COX-2 analgesics that are suitable for the present invention include those described in U.S. Pat. Nos. 5,474,995; 5,691,324; 6,063,811; 6,239,173; 5,466,823; 5,563,165; 5,760,068; and 5,972,986.

Preferred COX-2 analgesics include rofecoxib (MK-0966, MK-966, (4-[4-(methylsulfonyl)phenyl]-3-phenyl-2(5H)-furanone) (Chemical Abstracts Registry No.: 162011-90-7); L-745337; celecoxib (SC-58635, YM-74177, YM-177 (4-[5-(4-methylphenyl-3-(trifluoromethyl)-1H-pyrazol-1-yl]benzenesulfonamide (Chemical Abstracts Registry No.: 184007-95-2); parecoxib; valdecoxib; lumiracoxib; LAS 34475; and etorixocib.

In a particular embodiment, the present invention is directed to a method of treating or preventing pain, comprising administering to a subject in need of such treatment or prevention an analgesic selected from the consisting of rofecoxib, paracoxib, celecoxib, and valdecoxib; and albuterol, wherein said subject experiences an enhanced analgesic effect.

Opioids

The opioid analgesics (also known as opiates or narcotics) interact with a complex system of opioid receptors. The three major receptor classes are μ (mu), κ (kappa), and δ (delta). The μ receptor family mediates the actions of most clinically used opioids, including morphine, meperidine, propoxyphene, methadone, and fentanyl. The κ receptors are activated by the endogenous peptides dynorphins and may mediate some of the effects certain clinically effective opioids, including pentazocine, levorphanol, and nalbuphine. Highly-selective κ drugs have been developed but have not been clinically useful because of side effects such as dysphorias and dieresis. The δ receptors are activated by the endogenous enkephalin and may be useful targets for analgesic agents.

Opioids remain the drug of choice for moderate to severe acute pain. Unlike other analgesics, opioids do not display ceiling effects with regard to analgesia. However, opioids do exhibit a number of severe side effects. Such sided effects include dependence, tolerance, respiratory sedation, and gastrointestinal disturbances.

According to the present invention, an opioid analgesic is administered with a beta adrenergic agonist to treat or prevent pain.

Preferred opioid compounds useful in the present invention include anileridine, buprenorphine, butorphanol, codeine, dezocine, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, propiram (FBA-4503 (N-[1-methyl-2-(1-piperidinyl)ethyl)]-N-2-pyridinylproponamide, (E)-2-butenedioate (1:1) (Chemical Abstracts Registry No.: 13717-04-9 and 15686-91-6); and their pharmaceutically acceptable salts.

In one embodiment, opioid compounds are selected from the group consisting of morphine, hydromorphone, codeine, fentanyl, hydrocodone, levorphanol, meperidine, buprenorphine, butorphanol, nalbuphine, pentazocine, oxymorphone, and oxycodone.

In another embodiment, opioid compounds are selected from the group consisting of oxycodone, methadone, morphine, hydrocodone, hydromorphone, levorphanol, and codeine. In yet another preferred embodiment, opioid compounds are selected from morphine, oxycodone, hydromorphone, hydrocodone, and codeine.

Other opioid analgesics include opioid peptides such as orphanin FQ (also called nociceptin and OFQ/N). OFQ/N is an endogenous 17-amino acid peptide (FGGFTGARKSARKLADQ) that binds to the ORL-1 receptor but not the μ, k, or d receptors. Administration of OFQ/N produces analgesia. Additionally, other peptides related to OFQ/N produce analgesia. Such related peptides include OFQ/N(1-11) (FGGFTGARKSA), nocistatin (YEPGLEEVGEIEQKQLQ), OFQ2 (FSEFMRQYLVLSMQSSQ).

Other opioids not specifically mentioned herein are useful in the present invention. Opioids are well known in the art. The opioid activity of a compound can be readily determined by standard in vitro and in vivo potency and pharmacological assays that are well known in the art.

In a particular embodiment, the present invention is directed to a method of treating or preventing pain, comprising administering to a subject in need of such treatment or prevention an analgesic selected from the consisting of morphine, hydromorphone, codeine, fentanyl, hydrocodone, and oxycodone; and albuterol, wherein albuterol produces an enhanced activity of said analgesic.

In another embodiment, the present invention is directed to a method of treating or preventing pain, comprising administering to a subject in need of such treatment or prevention morphine and albuterol, wherein said subject experiences an enhanced analgesic effect.

Tramadol

Tramadol is a centrally acting synthetic opioid analgesic with two complementary mechanisms: 1) binding of parent and M1 metabolite to μ-opioid receptors and 2) inhibition of reuptake of norepinephrine and serotonin. Tramadol has demonstrated efficacy in a variety of conditions, including acute pain, neuropathic pain, arthritis, cancer pain and chronic pain. Although adverse effects from tramadol can be related to its effects through its dual mechanisms of action, like other opioids, tramadol produces dose related adverse effects, including dizziness, vertigo, nausea, vomiting, constipation, headache, somnolence. When given in high doses, tramadol can precipitate respiratory depression, which can have serious and potentially life threatening consequences.

In a particular embodiment, the present invention is directed to a method of treating or preventing pain, comprising administering to a subject in need of such treatment or prevention tramadol and albuterol, wherein said albuterol produces an enhanced effect of said tramadol.

Acetaminophen

Acetaminophen is an effective and widely used analgesic and antipyretic agent. Unlike NSAIDs, it is devoid of anti-inflammatory actions. In clinical trials comparing acetaminophen with NSAIDs in acute pain, maximal doses of acetaminophen are usually inferior to maximal doses of NSAID, in terms of magnitude of analgesia. The precise mechanism(s) of action of acetaminophen are not clear; however, acetaminophen is known to produce analgesia by elevation of the pain threshold and antipyresis through action on the hypothalamic heat-regulating center. Although short-term acetaminophen is generally safe, chronic heavy alcohol users are at increased risk of liver toxicity from excessive acetaminophen use. Acetaminophen has also been implicated in dose-related, life threatening liver toxicity in patients who do not have a history of heavy alcohol use. In addition, long-tem acetaminophen use has been implicated in dose-related chronic renal failure.

In particular embodiment, the present invention is directed to a method of treating or preventing pain, comprising administering to a subject in need of such treatment or prevention acetaminophen and albuterol, wherein said albuterol produces an enhanced effect of said acetaminophen.

In practicing the present invention, the analgesic may be in the form of a racemic mixture, unequal mixtures of stereiomers, a mixture of stereoisomers containing substantially more of one stereoisomer that other stereoisomers, or a substantially pure single stereoisomer.

In another embodiment, the present invention is directed to a method of treating or preventing pain, comprising administering to a subject in need of such treatment or prevention albuterol and morphine. In another embodiment, the albuterol and morphine are administered together in a single composition. In another embodiment, the albuterol and morphine are administered separately. With respect to dosage amounts, in one embodiment, morphine is administered in an amount of from about 1, 2, 3, 4, 5, 6, 8, 10, 15, 20, 25, 30, 35, 40, 45, and 50 mg. The morphine can be administered in any suitable form, e.g., morphine free base and morphine sulfate, and can be administered by any suitable route of administration, e.g., oral, i.v., etc. In one embodiment, the albuterol is administered in an amount sufficient to produce an enhanced activity of the morphine.

Types of Pain

The methods and compositions of the present invention are useful in treating all types of pain. Preferred types of pain to be treated by the present invention are neuropathic pain including diabetic neuropathy, shingles, postherpetic neuralgia, trigeminal neuralgia, nerve injury, spinal pain, stamp pain, phantom limb pain, and temporomandibular joint disorder; cancer pain; chronic pain; acute pain; breakthrough pain; low back pain; postsurgical pain; rheumatoid arthritis; osteoarthritis; headache, myofascial pain, fibromyalgia, sympathetically mediated pain, Raynaud's disease, CPS (Chronic Pain Syndrome); tension and migraine headache.

Chronic or intractable pain is often endured over many years or decades. Patients suffering from chronic pain often develop emotional problems which can lead to depression and in the worst case, attempted suicide. Long lasting pain often occurs particularly in joints, in muscles, connective tissue (e.g., fibromyalgia) and in the back. In the United States alone, chronic pain causes a loss of more than 250 million working days per year.

A patient is usually considered to have chronic pain when complaints thereof last longer than three months. In the course of time, chronic pain can come completely to the fore and form an independent clinical syndrome. Today most of the clinical phenomena of chronic pain syndrome are explained as a permanent excitation of spinal convergence neurons. This excitation can be provoked by either visceral or somatic afferent stimulation.

Certain types of pain have complex etiologies. For example, neuropathic pain is generally a chronic condition attributable to injury or partial transection of a peripheral nerve. This type of pain is characterized by hyperesthesia, or enhanced sensitivity to external noxious stimuli, allodynia, or application of an otherwise non-noxious stimuli, and paroxysmal pain. The hyperesthetic component of neuropathic pain does not respond to the same pharmaceutical interventions as does more generalized and acute forms of pain.

Neuropathic pain is a form of chronic pain that can persist for months, years, or decades following an injury and results from damage to peripheral nerves, nerve roots, the spinal cord, or certain brain regions. It differs from nociceptive pain in terms of duration, characteristics, underlying mechanisms and treatment (Bennett, G. J., in Textbook of Pain, Wall, P D and Melzack, R, eds., Churchill Livingstone, London 3rd edn, (1994a), pp. 201 et seq.). Neuropathic pain can consist of spontaneous pain (e.g., burning, cutting, tingling), evoked pain (e.g., allodynia evoked by stimulation of non-nociceptive afferents, and hyperalgesia evoked by stimulation of nociceptive afferents and paroxysmal pain (e.g., originating from a trigger point, described as stabbing, lancinating, shocklike) (Bennett, G. J., in Textbook of Pain, Wall, P D and Melzack, R, eds., Churchill Livingstone, London 3rd edn,(1994a), pp. 201 et seq.). Neuropathic pain can accompany nociceptive pain, and multiple treatment strategies may be required for optimal alleviation of pain (Portenoy, R. K., in Towards a New Pharmacology of Pain, Basbaum, A. I. and Besson, J. M., eds., John Wiley & Sons Ltd, New York (1991), pp. 393 et seq.; Devor M, et al., in Towards a New Pharmacotherapy of Pain, Basbaum, A. I. and Besson, J. M., eds., John Wiley & Sons, New York (1991), pp. 417 et seq.).

Other pain syndromes believed to have a neuropathic component are stump pain, fibromyalgia, myofascial pain, polyarteritis nodosa, osteomyelitis, bums involving nerve damage, AIDS related pain syndromes, and connective tissue disorders, such as systemic lupus erythematosis, systemic sclerosis, polymyositis, and dermatomyositis, and the like.

Acute pain is usually a consequence of an identifiable insult, such as surgery or other trauma, or a consequence of a disease, e.g., kidney stones, mechanical low back pain, etc. It may be treated with parenteral and oral opioid analgesics, NSAIDs, and more recently, COX-2 inhibitors. Recent surveys have suggested that the management of acute post-surgical pain may be inadequate due in part to dose-related side effects of opioids (e.g., nausea, vomiting, sedation, constipation and rare but potentially fatal respiratory arrest, shock, and cardiac arrest) and NSAIDs (e.g., gastrointestinal bleeding).

Administration

Administration of the analgesic and beta adrenergic agonist can be via oral, subcutaneous, intravenous, intramuscular, intraperitoneal, transdermal, transmucosal, buccal, inhalation, intranasal, epidural, intrathecal, intra-atricular, rectal or ocular routes.

The analgesic and the beta adrenergic agonist may be administered as a single pharmaceutical composition. That is, the analgesic and the beta adrenergic agonist may be formulated together so that both active agents are contained in one pharmaceutical compositions. For example, the analgesic and the beta adrenergic agonist may be formulated together in a unit dosage form. Suitable unit dosage forms include tablets, pills, capsules, caplets, hard gelatin capsule in powder or granular form, trochet, soft gelatin capsule.

While the analgesic and beta adrenergic agonist need not be administered together, they must both be present in the patient at effective levels at the same time. While it is within the scope of the invention to separately administer the analgesic and the beta adrenergic agonist, as a matter of convenience, in one embodiment, these drugs are coadministered in a single dosage form. All modes of co-administration are contemplated, e.g., orally, rectally, parenterally, topically, transdermally or by intravenous, intramuscular, intrastemal or subcutaneous injection or in a form suitable by inhalation. The formulations can, where appropriate, be conveniently presented in discrete dosage units and can be prepared by any of the methods well known in the art of pharmacy.

The methods of the present invention comprise administering an analgesic and administering a beta adrenergic agonist. Accordingly, the analgesic and the beta adrenergic agonist may be administered concurrently. When administered concurrently, the analgesic and the beta adrenergic agonist are administered to the subject at the same time. Concurrent administration may comprise administration of a single pharmaceutical composition comprising the analgesic and a beta adrenergic agonist. For example, a tablet comprising an analgesic and the beta adrenergic agonist is administered to a subject in need of analgesic treatment. Alternatively, concurrent administration may comprise administering of a first pharmaceutical composition comprising an analgesic agent and administering a second pharmaceutical composition comprising a beta adrenergic agonist.

In another embodiment, the method of the present invention comprises patient controlled analgesia. PCA is a technique for providing pain relieving medicine to patients. Most commonly, it refers to intravenous, epidural, or subcutaneous administration of an analgesic via a pumping device with the patient having some ability to control the timing and quantity of drug delivery. See, for example, U.S. Pat. No. 6,010,483. Pumps currently used for PCA generally give the clinician two parameters to set when prescribing a given drug for a patient. These include (1) a demand dose or bolus amount of drug administered whenever the patient presses a button and (2) a lockout interval which determines how soon after a bolus is administered a second bolus will be delivered if the patient pushes the button again. If a patient presses the button before the lockout interval has elapsed, the PCA pump simply ignores the request. The dose and lockout are programmed into the pump for an individual patient and drug combination. The dose is prescribed based on the clinician's assessment of the patient's opioid requirement (depending on weight, habituation, or other factors). The lockout interval is generally set depending on the time to onset of clinical effect of a given drug. The lockout interval is used to prevent a patient from giving himself or herself another bolus before the previous bolus has had a chance to take effect.

Dosage Levels

In general, the beta adrenergic agonist is administered according to the present invention at the lowest dose which produces an enhanced effect of the analgesic. In one embodiment, the method comprises administering a selected analgesic and a beta adrenergic, wherein said analgesic is administered at a subanalgesic dose. By subanalgesic dose, it is meant that the dose of analgesic administered in the method is lower than the dose of the same analgesic required to produce an analgesic effect when administered alone. Such a subanalgesic dose is understood by one of skill in the art.

In one embodiment, the method comprises administering a selected analgesic and a beta adrenergic, wherein said beta adrenergic is administered at a subanalgesic dose. By subanalgesic dose, it is meant that the dose of beta adrenergic administered in the method is lower than the dose of the same beta adrenergic required to produce an analgesic effect when administered alone. Such a subanalgesic dose is understood by one of skill in the art.

In adult humans, the dose of opioid required varies considerably, depending on type of pain (e.g., acute vs. advanced cancer), pain severity, tolerability of side effects, tolerance to analgesic effects, clinician's experience with the analgesic and patient response to treatment. There are established standards to allow practitioners of the art to convert the dose of one opioid analgesic, given by any route of administration, to an equivalent dose of another opioid analgesic, given by any route of administration. These analgesic conversion tables provide what in the art is called “analgesic equivalence” or “equianalgesic doses.” (See for example “Anon. Principles of Analgesic Use in the Treatment of Acute Pain and Cancer Pain,” Fourth Edition, 199, American, Pain Society, 1999; Gutstein H B & Akil H. “Opioid Analgesics.” In: Goodman and Gilman's The Pharmacologic Basis of Therapeutics, 10th Ed., HardmanJG & Limbird LE (Eds), p 569-619, McGraw-Hill, New York, N.Y.).

In one embodiment, the method comprises administering doses of narcotic analgesic/beta adrenergic agonist compositions, to be administered about every 4 to 6 hours as needed at doses of equivalent to oral morphine about 5 mg to about 120 mg, oral oxycodone about 2.5 to about 60 mg, oral hydromorphone about 0.5 to about 24 mg, parenteral morphine about 0.5 to about 24 mg, parenteral hydromorphone about 0.2-10 mg. When given as extended or sustained release oral formulations, the daily dose will usually be to 2 to 4 times the dose range provided above. When the opioid is given by another route of administration, (e.g., transdermal buprenorphine, transdermal fentanyl, intranasal morphine), the dose of narcotic analgesic in the composition is the analgesic equivalent of the doses provided here, when converted using standard analgesic equivalent tables, adjusted for differences in dosing frequency (e.g., for 24 hour (once a day) patch, the dose of the analgesic in the composition would first be converted to the analgesic equivalent dose expressed in mg of morphine, hydromorphone or oxycodone, then divided by 4 to 6). For humans, in one embodiment, an average unit dosage analgesic composition typically contains from about 0.001 mg to about 400 mg every 4 to 6 hours and about from 0.0001 mg/kg/day to 40 mg/kg/day of one or more beta adrenergic agonists to provide the claimed effect.

In another embodiment, the beta adrenergic agonist is administered in a dose of about 0.0001 mg/kg/day to 40 mg/kg/day. In another embodiment, the beta adrenergic agonist is administered in a dose of about 0.25 mg/kg to about 20 mg/kg. In another embodiment, the beta adrenergic agonist is administered in a dose of about 0.25 mg/kg, about 1 mg/kg, about 5 mg/kg, or about 20 mg/kg. In another embodiment, the beta adrenergic agonist is administered in a dose of about 2 mg/kg, about 4 mg/kg, about 8 mg/kg, or about 16 mg/kg.

In adult humans, the dose of NSAIDs and COX-2 inhibitors varies, depending on type of pain, pain severity, tolerability of side effects, tolerance to analgesic effects, experience with the analgesic and patient response to treatment. In one aspect of the invention, the method comprises administering a dose of a selected analgesic and a dose of beta adrenergic agonist, to be administered about every 4 to 24 hours. In another embodiment, the doses administered are as shown below in Table 1.

Dosage ranges which illustrate the present invention are show as follows.

TABLE 1
Opioid and Beta Adrenergic Agonist Combination

| | Amount of Opioid | | |
|---|---|---|---|
| | Per Dose (for | | Amount of |
| | daily dose, usually multiply | | Beta Adrenergic |
| | each amount by 2 to 4) | | Agonist |
| Opioid Analgesics | Oral (mg) | Parenteral (mg) | Per Dose (mg) |
| Anileridine | 10 to 75 | 10 to 75 | 0.001 to 400 |
| Buprenorphine | 0.15 to 8 | 0.15 to 8 | 0.001 to 400 |
| Butorphanol | 0.5 to 10 | 0.5 to 10 | 0.001 to 400 |
| Codeine | 15 to 120 | 15 to 60 | 0.001 to 400 |
| Dezocine | 1.5 to 30 | 3 to 30 | 0.001 to 400 |
| Diamophine | 1 to 20 | 1 to 20 | 0.001 to 400 |
| Dihydrocodeine | 10 to 120 | 10 to 120 | 0.001 to 400 |
| Fentanyl | — | 0.05 to 0.25 | 0.001 to 400 |
| Hydrocodone | 2.5 to 120 | 2.5 to 120 | 0.001 to 400 |
| Hydromorphone | 0.5 to 24 | 0.2 to 10 | 0.001 to 400 |
| | mg | | |
| Levorphanol | 0.5 to 10 | 0.25 to 8 | 0.001 to 400 |
| Meperidine | 25 to 150 | 25 to 150 | 0.001 to 400 |
| Meptazinol | 100 to 400 | 50 to 200 | 0.001 to 400 |
| Methadone | 2 to 30 | 2 to 30 | 0.001 to 400 |
| Morphine | 5 to 120 | 0.5 to 24 | 0.001 to 400 |
| Nalbuphine | — | 5 to 40 | 0.001 to 400 |
| Oxycodone | 2.5 to 60 | 0.5 to 20 | 0.001 to 400 |
| Oxymorphone | 5 to 60 | 0.5 to 5 | 0.001 to 400 |
| Pentazocine | 25 to 100 | 25 to 100 | 0.001 to 400 |
| Propiram | 25 to 150 | 25 to 150 | 0.001 to 400 |
| Propoxyphene | 65 to 200 | — | 0.001 to 400 |
| Tramadol | 25-400 | 25-400 | 0.001 to 400 |

TABLE 2
Beta Adrenergic Agonist Administered with
NSAID or COX-2 Inhibitor

| | | Amount of Beta |
|---|---|---|
| NSAID and COX-II | Amount of NSAID or COX-II | Adrenergic Agonist |
| Inhibitor Analgesics | Inhibitor Dose (mg) | Per Dose (mg) |
| Aspirin | 300 to 4000 | 0.001 to 400 |
| Celecoxib | 50 to 600 | 0.001 to 400 |
| Diclofenac | 12.5 to 200 | 0.001 to 400 |
| Diflusinal | 125 to 3000 | 0.001 to 400 |
| Etodolac | 50 to 1200 | 0.001 to 400 |
| Etoricoxib | 5 to 120 | 0.001 to 400 |
| Fenoprofen | 100 to 2400 | 0.001 to 400 |
| Flurbiprofen | | 0.001 to 400 |
| Ibuprofen | 100 to 3200 | 0.001 to 400 |
| Indomethacin | 12.5 to 200 | 0.001 to 400 |
| Ketoprofen | 12.5 to 300 | 0.001 to 400 |
| Ketorolac | 5 to 120 | 0.001 to 400 |
| Lumiracoxib | 50 to 600 | 0.001 to 400 |
| Meclofenamate Na | 25 to 400 | 0.001 to 400 |
| Mefenamic acid | 125 to 2000 | 0.001 to 400 |
| Meloxicam | 7.5 to 30 | 0.001 to 400 |
| Nabumetone | 250 to 2000 | 0.001 to 400 |
| Naproxen | 125 to 2000 | 0.001 to 400 |
| Oxaprozin | 300 to 1800 | 0.001 to 400 |
| Parecoxib | 10 to 120 | 0.001 to 400 |
| | mg | |
| Phenylbutazone | | 0.001 to 400 |
| Piroxicam | 5 to 40 | 0.001 to 400 |
| Rofecoxib | 7.5 to 100 | 0.001 to 400 |
| Salsalate | 300 to 4000 | 0.001 to 400 |
| Salicylate | 250 to 3000 | 0.001 to 400 |
| Sulindac | 25 to 400 | 0.001 to 400 |
| Tolmetin | 100 to 1800 | 0.001 to 400 |
| Valdecoxib | 2.5 to 40 | 0.001 to 400 |

In a further example, acetaminophen is administered at a dose of about 300 mg to about 1000 mg every four to six hours, 300 mg to 4000 mg per day, or from 3 mg/kg/day to about 70 mg/kg/day, when administered according to the present invention.

Compositions and Formulations

Another aspect of the present invention is a pharmaceutical composition comprising an analgesic composition containing the selected analgesic and beta adrenergic agonist. The analgesic composition is ordinarily formulated with one or more pharmaceutically acceptable ingredients (excipients and/or carriers) in accordance with known and established practice. Thus, the analgesic composition can be formulated as a liquid, powder, elixir, injectable solution, etc. Formulations for oral use can be provided as tablets or hard capsules wherein the pharmacologically active ingredients are mixed with an inert solid diluent such as calcium carbonate, calcium phosphate or kaolin, or as soft gelatin capsules wherein the active ingredients are mixed with water or miscible solvents such as propylene glycol; polyethylene glycols and ethanol, or an oleaginous medium, e.g., peanut oil, liquid paraffin or olive oil.

For topical administration in the mouth, the analgesic compositions can take the form of buccal or sublingual tablets, drops or lozenges formulated in conventional manner.

For topical administration to the epidermis the compounds of the invention can be formulated as creams, gels, ointments or lotions or as transdermal patches. Such compositions can, for example, be formulated with an aqueous or oily base with the addition of suitable thickening, gelling, emulsifying, stabilizing, dispersing, suspending, and/or coloring agents.

The compositions of the invention can also be formulated as depot preparations. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example as a sparingly soluble salt.

The compositions of the invention can be formulated for parenteral administration by injection, conveniently intravenous, intramuscular or subcutaneous injection, for example by bolus injection or continuous intravenous infusion. Formulations for injection can be presented in unit dosage from e.g., in ampoules, single-dose containers, or multi-dose containers, with or without added preservative(s). The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g. sterile pyrogen-free water, physiologic saline or dextrose 5% in water before use.

The compositions of the invention can also be formulated in rectal compositions such as suppositories or retention enemas, e.g., containing conventional suppository bases such as cocoa butter or other glyceride.

For intranasal administration, the compositions of the invention can be used, for example, as a liquid spray, as a powder or in the form of drops.

For administration by inhalation, the compounds according to the invention are conveniently delivered in the form of an aerosol spray presentation, solution or powder from pressurized packs or a nebulizer, with the use of a suitable propellant, e.g., dichlorodifluoromethane, trichlorofluoromethane, dichlorotetrafluoroethane, tetrafluoroethane, heptafluoropropane, carbon dioxide or other suitable gas, or without propellant. In the case of a pressurized aerosol the dosage unit can be determined by providing a valve to deliver a metered amount. Capsules and cartridges of, for example, gelatin, for use in an inhaler or insulator can be formulated containing a powder mix of a compound of the invention and a suitable powder base such as lactose or starch.

Aqueous suspensions can include pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. The aqueous suspensions can also contain one or more preservatives, e.g., ethyl- or n-propyl-p-hydroxybenzoate, one or more coloring agents, one or more flavoring agents and one or more sweetening agents, such as sucrose, saccharin or sodium or calcium cyclamate.

In an additional embodiment, in addition to the selected analgesic and beta adrenergic agonist, the composition according to the present invention herein comprises at least one other pharmacologically active substance, e.g., a non-narcotic analgesic such as acetaminophen, aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, tramadol, zomepirac, and the like or an opioid analgesic such as codeine, dihydrocodeine, hydrocodone, levorphanol, morphine, oxycodone, and the like.

Suitable formulations for parenteral administration include aqueous solutions of the active compounds in water-soluble form, for example, water-soluble salts, alkaline solutions and cyclodextrin inclusion complexes. One or more modified or unmodified cyclodextrins can be employed to stabilize and increase the water solubility of compounds of the present invention. Useful cyclodextrins for this purpose are disclosed in U.S. Pat. Nos. 4,727,064, 4,764,604, and 5,024,998.

In addition, suspensions comprising an analgesic and a beta adrenergic agonist, as appropriate oily injection suspensions, can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example, sodium carboxymethyl cellulose, sorbitol, and/or dextran. Optionally, the suspension may also contain stabilizers.

In addition, the combinations may be incorporated into biodegradable polymers allowing for sustained release of the compound, the polymers being implanted in the vicinity of where drug delivery is desired, for example, at the site of a tumor. The biodegradable polymers and their use are described, for example, in detail in Brem et al., J. Neurosurg. 74:441-446 (1991). The dosage administered will be dependent upon the age, health, and weight of the recipient, kind of concurrent treatment, if any, frequency of treatment, and the nature of the effect desired.

Depending upon the dosage form employed, the compositions of this invention may also contain other adjuvants that may be useful in formulating the particular dosage form or in its administration. Thus, for example, when administered as a tablet the products of this invention may also contain lubricants, excipients, binding agents, disintegrating agents, and flavoring agents. In addition these products may also contain other pharmaceutically active ingredients such as decongestants, analgesic adjuvants, antihistamines, expectorants, antitussives, diuretics, other analgesics, other anti-inflammatory agents, other antipyretics, other antirheumatics, antioxidants, vasodilators, smooth muscle relaxants, skeletal muscle relaxants, bronchodilators, vitamins, trace minerals, amino acids, and biological peptides.

Determination of Analgesic Activity

The analgesic effects of the compositions of the present invention can be evaluated in one or more of the tests described below:

Rat Tail Flick Test

The tail flick test was first described by D'Amour and Smith (1941), and remains essentially unchanged in application. (See generally D'Amour, F. E. and Smith, D. L., “A method for determining loss of pain sensation”, J. Pharmacol. Exp. Therap., 72:74-79(1941); Dewey, D. L. and Harris, L. S., The Tail-flick test. In: S. Ehrenpreis and A. Neidle (Eds.), Methods in Narcotic Research, Marcel Dekker, Inc., New York, 1975, pp. 101-109; and Dubner, R. and Ren, K., “Assessing transient and persistent pain in animals.” In: P. D. Wall and R. Melzack (Eds.), Textbook of Pain, Churchill Livingstone, London, 1999, pp. 359-369). Quite simply, the tail of a rat or mouse is exposed to radiant heat, and the latency to withdraw is determined. The basal heat intensity is set so that naive rats withdraw their tails within 2 to 3 sec. A cut-off latency of 10 sec (i.e., 3 to 4 times basal control value) is commonly employed to prevent tissue damage. An alternative to using radiant heat is to dip the tail into a water bath maintained at a fixed temperature, usually in the moderately noxious range of about 52° C. or 55° C. One advantage of a water bath is that the temperature is kept constant.

The tail-flick test is considered to be very robust in that weak analgesic agents are not detected by this test. In contrast, it is considered highly selective. There is a high degree of correlation between drugs that are identified as antinociceptive in the tail-flick test and clinically active analgesic agents. It is especially predictive of rank-order of potency of opioid-type analgesic agents, and the clinically effective dose of a novel opioid may be predicted by the relative potency of the drug to a known substance, such as morphine, based on this assay. Importantly, agents that are sedating and may produce a positive response in the writhing test or hot plate test do not show antinociceptive activity in the tail-flick test. It is even possible to perform the tail-flick test in lightly anesthetized animals.

Data obtained from the rat tail-flick test conform to a graded dose-response curve. The raw tail withdrawal latencies are converted to a % MPE (% maximal possible effect) by the formula:

% MPE=100×(test latency−basal latency)/(cut-off−basal latency).

This formula constrains the data to fit between 0% MPE and 100% MPE. This allows the generation of dose-response curves and the calculation of ED₅₀ values (50% effective doses) with attendant confidence intervals. These calculations then allow for the determination of relative potencies of different drugs and allow for the isobolographic determination of possible synergistic effects. Instances where the test latency is less than the basal latency produces a negative % MPE, which is meaningless unless one is measuring hyperalgesia. By convention, these values are set to 0% MPE when the expected drug effect is antinociception or no activity.

Kim and Chung Peripheral Neuropathy Model

The method of Kim and Chung is used to evaluate the potential analgesic properties of one or more compounds in a model of peripheral mononeuropathy. (See Kim, S. H. & Chung, J. M. “An experimental peripheral neuropathy produced by segmental nerve ligation in the rat,” Pain 50:355-363 (1992); Hargreaves, K., et al., “A new and sensitive method for measuring thermal nociception in cutaneous hyperalgesia,” Pain 32:77-88 (1988); Lynn, B. & Carpenter, S. E. “Primary afferent units from the hairy skin of the rat hind limb,” Brain Research 238:29-43 (1982)). Following baseline assessment of mechanical, thermal, and cold allodynia over 2 days, a peripheral mononeuropathy is induced by tight ligation of the left L5 and L6 spinal nerves under aseptic conditions. The animals are allowed to recover from surgery for six days before the behavioural testing is recommenced. Typically, behavioral testing is resumed on day 7 post-operatively and repeated on days 9 and 11 to monitor the development of allodynia and/or hyperalgesia. Tests with the putative analgesic(s) are carried out on or about day 12 (the time-point corresponding to maximal behavioural changes).

The following tests are typically performed in combination depending on the parameters to be assessed. A minimum period of 5 minutes is allowed between each type of test (or repeat challenges to the same paw) to reduce the risk of sensitization. Mechanical allodynia test: The animal is placed in a wire mesh cage, and a series of Von Frey filaments are applied to the plantar surface of the hind paw, from below. The filaments are applied in ascending order (starting with the weakest force), and the withdrawal threshold for both the ipsilateral and contralateral hind paws is evaluated. The withdrawal threshold is defined as being the lowest force of two or more consecutive Von Frey filaments to elicit a reflex withdrawal response (i.e., a brief paw flick). Thermal hyperalgesia test: Rats are placed in clear plastic chambers with a glass floor and allowed a short period to acclimatize to their environment prior to testing (approximately 2 minutes). The animals are then challenged with a radiant infrared heat source, directed at the plantar surface of their hind paw from below, and the withdrawal latency calculated for both the ipsilateral and contralateral hind paws. Cold allodynia test: Rats are placed on a metal platform submerged approximately 1 cm below the surface of iced water (approximately 4° C.), such that the hairy and glabrous skin of the animals feet are in contact with the cold water. Following an acclimatization period of approximately 30 seconds, the suspended paw elevation time (SPET) for the ipsilateral hind paw (that is, the total, accumulative length of time the animal holds its affected hind paw out of the water in a defensive posture) is measured during a 20 second challenge.

Standard statistical methods are employed to evaluate test substance related effects. Data are analyzed for homogeneity and either parametric or non-parametric methods applied as appropriate.

Carrgeenan-Induced Inflamed Paw Model

Models of inflammation that produce more persistent pain include the injection of carrageenan into the footpad of the limb; the potential analgesic and/or anti-inflammatory properties of putative analgesics substances can be evaluated in this model. See generally Bhalla T. N. & Tangri, K. K. “The time course of the carrageenan-induced oedema of the paw of the rat.” J. Pharm. Pharmacol. 22:721 (1970); Randall, L. O. & Selitto, J. J., “A method for measurement of analgesic activity on inflamed tissue,” Arch. Int. Pharmacodyn. Ther. 111:409-419 (1957); Hargreaves, K., et al. “A new and sensitive method for measuring thermal nociception in cutaneous hyperalgesia.” Pain 32:77-88 (1988).

Typically, rats are handled and acclimatized to the behavioral testing equipment over a minimum of 2 days prior to testing. Behavioral tests are performed on all rats on the day prior to dosing to establish baseline values, and the animals are randomized into treatment groups based on these pre-dose responses. An assessment of the inflammatory agent (carrageenan) is performed prior to the main study, using the chosen behavioral tests. On the day of dosing, an inflammatory response is induced in the left hind paw of each rat by an intraplantar injection (approx. 0.05 mL) of carrageenan (0.6% w/v), under brief anesthesia. The test substance, reference substance, or vehicle is generally administered 30 minutes prior to carrageenan administration for oral dosing.

The following tests may be performed. A minimum period of 5 minutes is allowed between each type of test (or repeat challenges to the same paw) to reduce the risk of sensitization.

Paw Volume: Each animal is gently restrained, their hind limb extended, and the paw placed in the pre-filled chamber of a Digital Plethysmometer. The paw volume is then calculated based on the volume of liquid displaced in the chamber, for both the ipsilateral and contralateral hind paws.

Mechanical hyperalgesia test: Each rat is gently restrained, their hind limb extended, and the paw placed lightly on the Randall-Selitto device. A progressively increasing pressure is then applied to the dorsal surface of the paw via a blunt peg attached to a weight level, and the withdrawal threshold calculated for both the ipsilateral and contralateral hind paws. The maximum pressure applied is about 250 g. The withdrawal threshold is defined as the minimum force (in grams) required to elicit a reflex withdrawal response. Typical end points are a struggle response, paw withdrawal or a squeak response.

Thermal hyperalgesia test: Rats are placed in clear plastic chambers with a glass floor and allowed a short period to acclimatize to their environment prior to testing (approximately 2-5 minutes). The animals are then challenged with a radiant infrared heat source, directed at the plantar surface of their hind paw from below, and the withdrawal latency calculated for both the ipsilateral and contralateral hind paw

Standard statistical methods are employed to evaluate test substance related effects. Data are analyzed for homogeneity and either parametric or non-parametric methods applied.

The included examples are illustrative but not limiting of the methods and composition of the present invention. Other suitable modifications and adaptations of the variety of conditions and parameters normally encountered and obvious to those skilled in the art are within the spirit and scope of the invention.

### EXAMPLES

### Evaluation of Synergy Between Albuterol (Salbutamol) and Morphine

Importantly, albuterol given alone produced a minimal antinociceptive effect over the dose range of 0.25 mg/kg to 20 mg/kg. Although the increases in tail-flick latency were statistically significant, these increases were only in the order of 1 to 1.2 sec, which is of limited biological significance, and are thus considered inactive this analysis. Absent any interaction, the dose-effect curves for morphine alone and in the presence of albuterol should be identical. Alternatively, a significant shift to the left of the dose-effect curve for morphine in the presence of albuterol would indicate a synergistic interaction.

In order to evaluate dose-effect curves and test for relative potency, the data were converted from the raw values into % maximal possible effect (% MPE) in order to produce responses ranging from 0% to 100%. This manipulation allowed the construction of dose-effect curves and more importantly, the calculation of the A₅₀ (dose required to produce a 50% effect) and the confidence intervals. The data were converted to % MPE based on the equation:

% MPE=(response−baseline)/(8.2−baseline)×100.

Baseline refers to the pre-treatment tail-flick latencies, and 8.2 represents the maximal effect. This value is derived from the 8.2 sec which is the largest response observed in this study, occurring 60 minutes after the injection of 5 mg/kg of morphine and 1 mg/kg of albuterol. Thus, this represented the ceiling effect, for the purposes of this analysis. The A₅₀ values and confidence intervals for each of the dose-effect curves were determined from linear regression analysis of the logdose-effect curves. Significant shifts in the dose-effect curves were indicated by Student's t-statistic applied to the A₅₀ values obtained for morphine and morphine plus albuterol, and was performed with our custom-designed data analysis software.

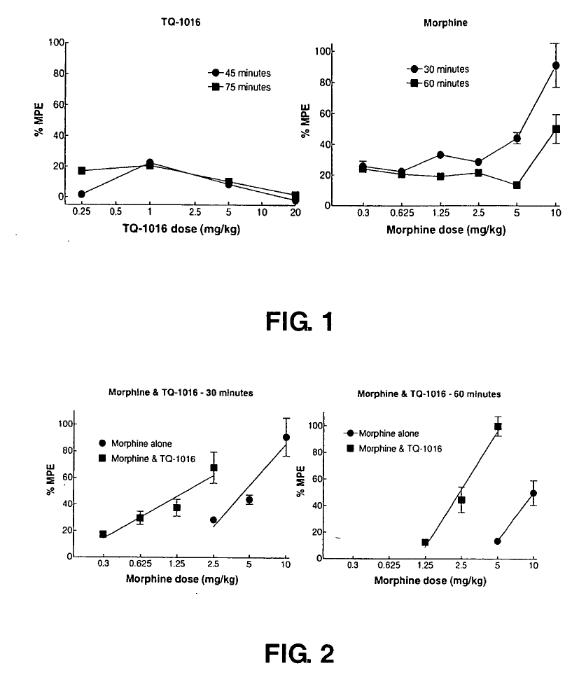

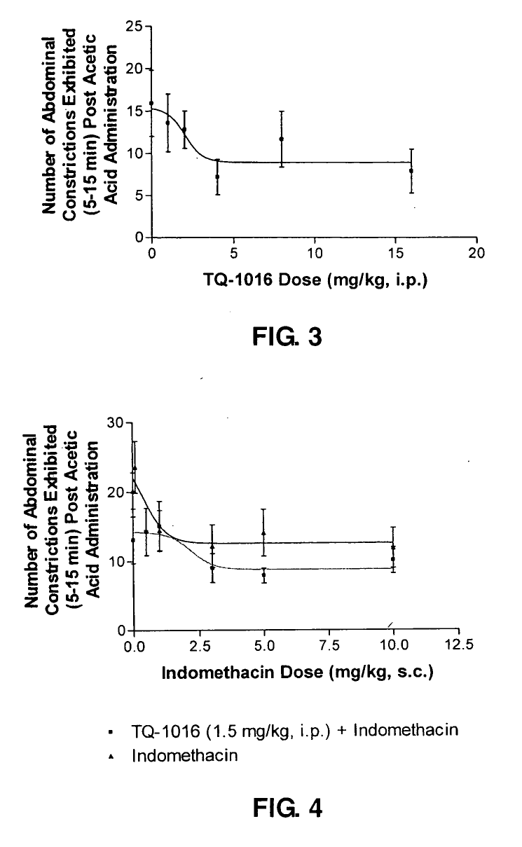

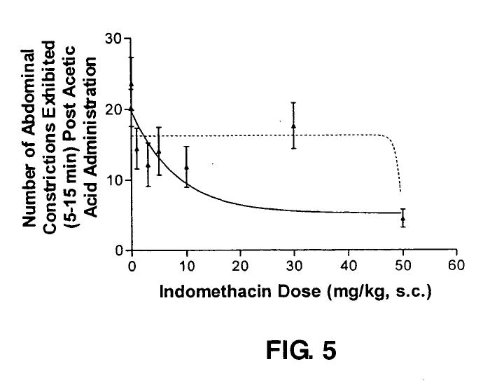

Table 3 shows the data for albuterol, and Table 4 shows the data for morphine converted to % MPE (FIG. 1). The SEM values are approximate and are derived as a ratio of the SEM of the raw data to the respective mean and adjusted as a function of % MPE.

TABLE 3
Antinociceptive Effect of Albuterol alone (as % MPE)

| Albuterol dose | Baseline | | |
|---|---|---|---|
| (mg/kg) | (sec) | 45 minutes | 75 minutes |
| 0.25 | 2.3 ± 0.1 | 1.69 ± 0.21 | 16.95 ± 1.54 |
| 1 | 2.4 ± 0.1 | 22.41 ± 1.82 | 20.69 ± 1.72 |
| 5 | 2.4 ± 0.1 | 8.62 ± 0.59 | 10.34 ± 1.03 |
| 20 | 2.5 ± 0.1 | −1.75 ± 0.15 | 1.75 ± 0.2 |

TABLE 4
Antinociceptive Effect of Morphine alone (as % MPE)

| Morphine dose | Baseline | | |
|---|---|---|---|
| (mg/kg) | (sec) | 30 minutes | 60 minutes |
| 0.3125 | 2.4 ± 0.2 | 25.86 ± 3.31 | 24.14 ± 1.27 |
| 0.625 | 2.4 ± 0.2 | 22.41 ± 1.82 | 20.69 ± 1.72 |
| 1.25 | 2.5 ± 0.2 | 33.33 ± 2.27 | 19.3 ± 2.14 |
| 2.5 | 2.6 ± 0.2 | 28.57 ± 2.04 | 21.43 ± 1.69 |
| 5 | 2.3 ± 0.1 | 44.07 ± 3.6 | 13.56 ± 2.19 |
| 10 | 2.6 ± 0.2 | 91.07 ± 14.19 | 50 ± 9.26 |

The calculated effect of albuterol did not rise above 20% MPE over the entire dose-range tested. This value indicates minimal antinociceptive effect and is generally considered to be biologically insignificant. Morphine given in doses of 0.3 mg/kg to 2.5 mg/kg also produced minimal effects, and 2.5 mg/kg represented the beginning of the linear portion of the dose-response curve 30 minutes after injection. The A₅₀ value for morphine 30 minutes after injection was based on the data between 2.5 mg/kg and 10 mg/kg, and was calculated to be 4.53 mg/kg (95% C.I.: 3.91 to 5.24). The A₅₀ value for morphine at 60 minutes was 9.94 mg/kg (95% C.I.: 9.84 to 10). The dose-effect curves for morphine alone were compared to those for morphine in the presence of albuterol at the 30- and 60-minute time points (FIG. 2). These dose-response curves were constructed from the linear portions of the effect curves. The enhanced analgesic effect of morphine in the presence of albuterol was synergistic at both time points. The A₅₀ value for morphine in the presence of albuterol 30 minutes after injection was 1.50 mg/kg (95% C.I.: 1.26 to 1.77). This represents a significant 3-fold shift to the left of the dose-response curve. Similarly, the A₅₀ value of morphine plus albuterol 60 minutes after injection was 2.41 mg/kg (95% C.I.: 2.24 to 2.61), representing a significant 4-fold shift to the left of the dose-response curve. If there were no enhanced analgesic effect of morphine in the presence of albuterol, then the A₅₀ values of morphine in the presence of albuterol would be the same as those of morphine alone. These results are summarized in Table 5 below. A significant potency ratio is indicated when the confidence intervals of the potency ratio exclude unity (Tallarida and Murray, 1987).

TABLE 5
Summary of Dose-response Data and Potency Ratio

| A₅₀ (95% | | |
|---|---|---|
| confidence | | |
| interval) | 30 minutes | 60 minutes |
| Morphine alone | 4.53 (3.92 to 5.24) | 9.94 (9.84 to 10.04) |
| r² | 0.9231 | 1.00 |
| slope | 104 | 123 |
| Morphine & | 1.4969 (1.26 to 1.77) | 2.42 (2.24 to 2.61) |
| Albuterol | | |
| r² | 0.9158 | 0.9771 |
| Slope | 52.8 | 145 |
| Potency ratio | 3.02 (2.42 to 3.78) | 4.1126 (3.80 to 4.45) |
| | Significant | Significant |

The data show that albuterol alone has no biologically significant antinociceptive activity. Under the conditions of the experiments, there was a synergistic interaction between albuterol and morphine. Co-administration of albuterol with morphine increased the analgesic potency of morphine 3 to 4 fold.

TABLE 6
Effect Of Albuterol On Chemically-Induced
Abdominal Constrictions In The Mouse

Number of Abdominal
Constrictions
Exhibited in
Time Period (min)
Post Acetic Acid

| Group | Treatment (i.p.) | 5-10 | 5-15 |
|---|---|---|---|
| Q | Vehicle 1 | 8.1 ± 2.0 | 15.9 ± 3.9 |
| (n = 10) | (2 ml/kg) | | |
| D | Albuterol | 8.4 ± 2.3 | 13.6 ± 3.4 |
| (n = 10) | (1 mg/kg) | | |
| B | Albuterol | 5.2 ± 1.6 | 12.8 ± 2.2 |
| (n = 10) | (2 mg/kg) | | |
| E | Albuterol | 3.8 ± 1.4 | 7.2 ± 2.1 |
| (n = 10) | (4 mg/kg) | | |
| A | Albuterol | 5.7 ± 1.7 | 11.7 ± 3.3 |
| (n = 10) | (8 mg/kg) | | |
| C | Albuterol | 3.4 ± 1.4 | 7.9 ± 2.6 |
| (n = 10) | (16 mg/kg) | | |

Data are expressed as mean ± S.E.M.
Vehicle 1 for Albuterol was 0.9% w/v sodium chloride.
Albuterol or vehicle 1, were dosed 25 min prior to acetic acid (0.7% v/v) administration.

TABLE 7
Effect of Indomethacin on Chemically-Induced Abdominal
Constrictions in the Mouse

Number of Abdominal
Constrictions Exhibited
in Time Period (min)
Post Acetic Acid

| Group | Treatment (s.c.) | 5-10 | 5-15 |
|---|---|---|---|
| J | Vehicle 2 | 12.6 ± 1.7 | 20.2 ± 2.6 |
| (n = 10) | (10 ml/kg) | | |
| S | Indomethacin | 14.3 ± 2.6 | 23.6 ± 3.7 |
| (n = 10) | (0.1 mg/kg) | | |
| R | Indomethacin | 7.5 ± 2.0 | 14.5 ± 2.9 |
| (n = 10) | (1 mg/kg) | | |
| I | Indomethacin | 6.8 ± 1.7 | 12.2 ± 3.1 |
| (n = 10) | (3 mg/kg) | | |
| H | Indomethacin | 8.3 ± 2.0 | 14.1 ± 3.4 |
| (n = 10) | (5 mg/kg) | | |
| G | Indomethacin | 6.8 ± 1.7 | 11.9 ± 2.9 |
| (n = 10) | (10 mg/kg) | | |
| F | Indomethacin | 10.5 ± 2.3 | 17.6 ± 3.2 |
| (n = 10) | (30 mg/kg) | | |
| T | Indomethacin | 1.4 ± 0.6 | 4.5 ± 1.3 |
| (n = 10) | (50 mg/kg) | | |

All data are expressed as mean ± S.E.M.
Vehicle 2 for indomethacin was 0.5% w/v CMC and 0.5% v/v Tween 80.
Indomethacin or vehicle 2, were dosed 20 min prior to acetic acid (0.7% v/v) administration.

TABLE 8
Effect of Co-administration of Albuterol and Indomethacin on
Chemically-Induced Abdominal Constrictions in the Mouse

| | | Number of Abdominal | |
|---|---|---|---|
| | | Constrictions | |
| | | Exhibited in | |
| | | Time Period (min) | |
| | | Post Acetic Acid | |
| Group | Treatment | 5-10 | 5-15 |
| P | Vehicle 1 (2 ml/kg, i.p.) + Vehicle 2 | 6.3 ± 2.0 | 13.1 ± 3.4 |
| (n = 10) | (10 ml/kg, s.c.) | | |
| N | Albuterol (1.5 mg/kg) + Indomethacin | 6.4 ± 2.1 | 14.3 ± 3.4 |
| (n = 10) | (0.5 mg/kg) | | |
| M | Albuterol (1.5 mg/kg) + Indomethacin | 7.3 ± 2.1 | 15.1 ± 3.6 |
| (n = 10) | (1 mg/kg) | | |
| K | Albuterol (1.5 mg/kg) + Indomethacin | 3.9 ± 1.0 | 9.0 ± 2.1 |
| (n = 10) | (3 mg/kg) | | |
| L | Albuterol (1.5 mg/kg) + Indomethacin | 2.9 ± 0.8 | 7.9 ± 1.1 |
| (n = 10) | (5 mg/kg) | | |
| O | Albuterol (1.5 mg/kg) + Indomethacin | 3.7 ± 1.3 | 10.1 ± 1.9 |
| (n = 10) | (10 mg/kg) | | |

All data are expressed as mean ± S.E.M.
Vehicle 1 for Albuterol was 0.9% w/v sodium chloride.
Vehicle 2 for indomethacin was 0.5% w/v CMC and 0.5% v/v Tween 80.

Albuterol or vehicle 1 was dosed intraperitoneally (−25 min), followed by a subcutaneous dose of indomethacin or vehicle 2 (−20 min), prior to acetic acid (0.7% v/v) administration (0 min).

Having now fully described the invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

What is claimed is:

1. A method of treating or preventing pain comprising administering to a subject in need of pain treatment or pain prevention
(a) one or more analgesics, wherein said analgesic is selected from the group consisting of opioids, NSAIDs, COX-2 inhibitors, acetaminophen, and tramadol; and
(b) one or more beta adrenergic agonists, wherein said beta adrenergic agonist produces an enhanced effect of said analgesic,
provided that said enhanced effect of said analgesic does not include NSAID-induced gastrointestinal injury.

2. The method of claim 1, wherein said analgesic is administered prior to, the administration of said beta adrenergic agonist.

3. The method of claim 1, wherein said analgesic is administered after the administration of said beta adrenergic agonist.

4. The method of claim 1, wherein said analgesic is administered concurrently with said beta adrenergic agonist.

5. The method of claim 1, wherein said enhanced effect is a faster onset of action.

6. The method of claim 1, wherein said enhanced effect is an increased duration of action.

7. The method of claim 1, wherein said enhanced effect is a reduction of one ore more side effects of said analgesic.

8. The method of claim 1, wherein said effect is an increased maximal analgesic effect of said analgesic.

9. The method of claim 1, wherein said analgesic is administered in a subanalgesic amount.

10. The method of claim 1, wherein said beta adrenergic agonist is administered in a subanalgesic amount.

11. The method of claim 1, wherein said beta adrenergic agonist is administered in an amount sufficient to reduce analgesic tolerance.

12. The method of claim 1, wherein said beta adrenergic agonist is administered in an amount sufficient to reduce opioid dependence.

13. The method of claim 1, wherein said beta adrenergic agonist is administered in an amount sufficient to reduce side effects of said analgesic, wherein said analgesic is an opioid or acetaminophen.

14. The method of claim 1, wherein said analgesic is selected from the group consisting of COX-2 inhibitors, opioid, acetaminophen, and tramadol.

15. The method of claim 1, wherein said analgesic is a COX-2 inhibitor.

16. The method of claim 1, wherein said analgesic is an NSAID.

17. The method of claim 1, wherein said analgesic is an opioid.

18. The method of claim 1, wherein said analgesic is acetaminophen.

19. The method of claim 1, wherein said analgesic is tramadol.

20. The method of claim 1, wherein said beta adrenergic agonist is selected from the group consisting of bitolterol, broxaterol, cimaterol, clenbuterol, colterol, fenoterol, fomoterol, formoterol, isoetharine, isoproterenol (isoprenaline), isoxsuprine, mabuterol, metaproterenol, orciprenaline, picumeterol, procaterol, ractopamine, reproterol, rimiterol, ritodrine, salbutamol (albuterol), salmeterol, terbutaline, and zinterol.

21. The method of claim 20, wherein said beta adrenergic agonist is selected from the group consisting of albuterol, isoproteronol, and terbutaline.

22. The method of claim 21, wherein said beta adrenergic agonist is albuterol.

23. The method of claim 1, wherein said subject is a mammal.

24. The method of claim 23, wherein said mammal is a human.

25. The method of claim 1, wherein said analgesic and said beta adrenergic agonist are administered to said subject by a route selected from the group consisting of oral, subcutaneous, intravenous, intramuscular, topical, transdermal, transmucosal, buccal, inhalation, epidural, intrqathecal, rectal, intrarticular, and ocular.

26. The method of claim 25, wherein said beta adrenergic agonist is administered orally.

27. The method of claim 26, wherein said analgesic is administered orally.

28. The method of claim 1, wherein said analgesic and said beta adrenergic agonist are administered as a single pharmaceutical composition.

29. The method of claim 1, wherein said analgesic and said beta adrenergic agonist are administered as separate pharmaceutical compositions.

30. The method of claim 1, wherein said analgesic and said beta adrenergic agonist are coadministered as a sustained release dosage form.

31. The method of claim 1, wherein said beta agonist is administered in an amount of about 0.001 to 400 mg, preferably 0.01 mg to about 40 mg per dose, preferably 0.1 mg to about 4 mg per dose.

32. A composition comprising (a) an analgesic selected from the group consisting of a COX-2 inhibitor, opioid, NSAID, acetaminophen, and tramadol; and
(b) a beta adrenergic agonist.

33. The composition of claim 32, wherein said beta adrenergic agonist is in an amount which enhances the activity of said analgesic.

34. The composition of claim 32, wherein said beta adrenergic agonist is in an amount which enhances the activity of said analgesic.

35. The composition of claim 32, wherein said beta adrenergic agonist is in an amount which hastens the onset of activity of said analgesic.

36. The composition of claim 32, wherein said beta adrenergic agonist is in an amount which increases the duration of the activity of said analgesic.

37. The composition of claim 32, wherein said beta adrenergic agonist is selected from the group consisting of bitolterol, broxaterol, cimaterol, clenbuterol, colterol, fenoterol, fomoterol, formoterol, isoetharine, isoproterenol (isoprenaline), isoxsuprine, mabuterol, metaproterenol, orciprenaline, picumeterol, procaterol, ractopamine, reproterol, rimiterol, ritodrine, salbutamol (albuterol), salmeterol, terbutaline, and zinterol.

38. The composition of claim 37, wherein said beta adrenergic agonist is selected from the group consisting of albuterol, isoproteronol, and terbutaline.

39. The composition of claim 37, wherein said beta adrenergic agonist is albuterol.

40. The composition of claim 30, further comprising one or more excipients and optionally one or more inert carrier.
